(19) United States
(12) Patent Application Publication
(10) Pub. No.: US 2022/0126081 A1
(43) Pub. Date: Apr. 28, 2022

(54) PLASTIC TUBING KINK REMOVAL DEVICE AND METHOD

(71) Applicant: Hemanext Inc., Lexington, MA (US)

(72) Inventors: Jeffrey K. Sutton, Medway, MA (US); Austin Lent, Waltham, MA (US); George Michaels, Littleton, MA (US)

(73) Assignee: Hemanext Inc., Lexington, MA (US)

(21) Appl. No.: 17/418,682

(22) PCT Filed: Dec. 30, 2019
(86) PCT No.: PCT/US2019/068925

§ 371 Date: Jun. 25, 2021

Related U.S. Application Data
(60) Provisional application No. 62/787,606, filed on Jan. 2, 2019.

(51) Int. Cl. A61M 39/08; A61M 39/28; A61L 29/04; A61M 1/28; A61M 5/14; A61M 1/16

(52) U.S. Cl. CPC A61M 39/08; A61M 39/28; A61L 29/041; A61M 1/1621; A61M 2039/087; A61M 5/14; A61M 1/285

(57) ABSTRACT

This application provides methods, compositions, and kits for use in removing kinks in tubing.

### CROSS-REFERENCE TO RELATED APPLICATION

This application claims priority from U.S. Provisional Patent Application Ser. No. 62/787,606, filed Jan. 2, 2019, hereby incorporated by reference in its entirety.

### FIELD OF THE INVENTION

This invention relates to a device for the removal of kinks from plastic tubing for its use in transfusion medicine.

### BACKGROUND OF THE INVENTION

Plastic tubing is used for transferring fluid and is a component of fluid delivery and collection kits. Such tubing, typically made of polyvinylchloride (PVC), is subject to folds or kinks which results in an undesirable or unusable reduction of the inner lumen of the tubing for fluid flow. The use of plastic tubing clamps in delivery and collection kits (e.g., blood collection kits) can also causes the plastic tubing to kink during storage, thereby reducing or totally occluding the lumen of the tubing and preventing fluid flow. This problem is exacerbated with sterilization, and particularly so if the clamp is left in the closed position, either intentionally or accidentally.

In addition, it is common practice to utilize a Sterile Connection Device (“SCD”) to join different pieces of tubing. When the pieces of tubing are joined by the SCD, they typically have a section adhered together similar to a kink, which must be manipulated to restore fluid flow therein. Another source of kinking are folds in the tubing. The present invention overcomes the kinks and folds in order to restore the fluid flow in kinked sections of tubing.

### SUMMARY OF THE INVENTION

The present disclosure provides for, and includes, a device for restoring flow to a kinked tubing comprising a sleeve having a cylindrical inner surface that is larger than the outside diameter of the tubing and the sleeve is slide-able along the tubing to compress the kinked tubing perpendicular to the kink.

The present disclosure also provides for, and includes, a method of removing a kink in a tube comprising: sliding a sleeve comprising a cylindrical inner diameter along a tube.

The present disclosure further provides for, and includes, a device for restoring flow to a kinked tubing comprising a sleeve having a cylindrical inner surface equal to or smaller than the outside diameter of the tubing and capable of compressing the kinked tubing perpendicular to the kink.

### BRIEF DESCRIPTION OF THE DRAWINGS

The present disclosure is provided with reference to the accompanying drawings, wherein:

FIG. 1 illustrates a sleeve comprising an outside diameter (41), an inside diameter (42), a thickness (43), and a length (44), for removing kinks.

FIG. 2 illustrates a sleeve on a clamped tube according to Example 1.

FIGS. 3A and B illustrate a PVC tube with a kink according to Example 1.

FIGS. 4A and B illustrate a PVC tube with a kink removed using a sleeve according to Example 1.

FIGS. 5A and B illustrate an open sleeve with a living hinge smaller (A) or larger (B) than the longitudinal length of the sleeve.

FIG. 6 illustrates an exemplary embodiment of a sleeve as described herein.

FIGS. 7A to C illustrate exemplary embodiments of a (A) sleeve comprising (B) an hourglass shape or (C) cutouts.

FIGS. 8A to D illustrate exemplary embodiments of a sleeve (A) comprising an opening (B) being placed onto a kinked tube (C and D).

The example set out herein illustrates several embodiments of the present disclosure but should not be construed as limiting the scope of the present disclosure in any manner.

### DETAILED DESCRIPTION OF THE INVENTION

The present disclosure relates to, and includes, a sleeve having cylindrical inner surface for restoring flow to a kinked tubing. In an aspect of the present disclosure, the sleeve comprises a material selected from the group consisting of polyethylene (PE), polypropylene (PP), metal, polyethylene terephthalate (PETG), polyvinyl chloride (PVC), PC, acrylic, nylon, acrylonitrile-butadiene-styrene (ABS), polytetrafluoroethylene (PTFE or Teflon), polyvinylidene fluoride (PVDF), polyether ether ketone (PEEK), chlorinated polyvinyl chloride (CPVC), fluorinated ethylene propylene (FEP), perfluoroalkoxy alkanes (PFA), Polyimide, Acetal, polyetherimide, and Ultem. In another aspect, the sleeve comprises two or more materials selected from the group consisting of polyethylene (PE), polypropylene (PP), metal, polyethylene terephthalate (PETG), polyvinyl chloride (PVC), PC, acrylic, nylon, acrylonitrile-butadiene-styrene (ABS), polytetrafluoroethylene (PTFE or Teflon), polyvinylidene fluoride (PVDF), polyether ether ketone (PEEK), chlorinated polyvinyl chloride (CPVC), fluorinated ethylene propylene (FEP), perfluoroalkoxy alkanes (PFA), Polyimide, Acetal, polyetherimide, and Ultem. In another aspect, the sleeve comprises PVC.

In an aspect of the present disclosure, a sleeve comprises an inner cylindrical surface and an outer surface. In an aspect, the outer surface of the sleeve comprises a raised feature. In another aspect, the outer surface of the sleeve comprises a feature selected from the group consisting of stippling, ribbing, and knurling. In another aspect, the outer surface of the sleeve comprises a diamond knurling. In another aspect, the outer surface of the sleeve comprises a straight knurling. In a further aspect, the outer surface of the sleeve comprises a straight diagonal knurling. In another aspect, the inner surface comprises a chamfer.

In an aspect, the sleeve has an outer shape selected from the group consisting of a square, a triangle, a rectangle, a cylinder, a hexagon, an equilateral, a quadrilateral, a pentagon, an hourglass, and a trapezoid.

In an aspect of the present disclosure, the sleeve is semi-rigid. In another aspect, the sleeve is rigid. In another aspect, the sleeve is flexible. The rigidity of a polymer is described by flexural modulus (or modulus of elasticity in bending), which is the ratio of stress to strain of the polymer. In an aspect, the flexural strength of the sleeve is about 0.04, 0.07, 0.075, 0.08, 0.09, 0.1, 0.12, 0.14, 0.15, 0.16, 0.18, 0.2, 0.25, or 0.27 gigapascal (GPa). See, MatWeb, Flexural Strength Testing of Plastics (available on the internet at www(dot)matweb(dot)com/reference/flexuralstrength.aspx), and Omnexus, Fluxural Modulus or Bend Modulus, (available on the internet at omnexus(dot)specialchem(dot)com/polymer-properties/properties/stiffness). In another aspect the flexible modulus of the sleeve is between 1.0 and 3.0, 4.0 and 6.0, 2.0 and 4.0, 5.0 and 7.0, 3.0 and 5.0, 1.0 and 5.0, 5.0 and 9.0, 7.0 and 9.0, 0.0005 and 3.0, 4.0 and 7.0, 0.5 and 2.0, and 0.0005 and 1.0 gigapascal (GPa). Rigidity increases as GPa increases.

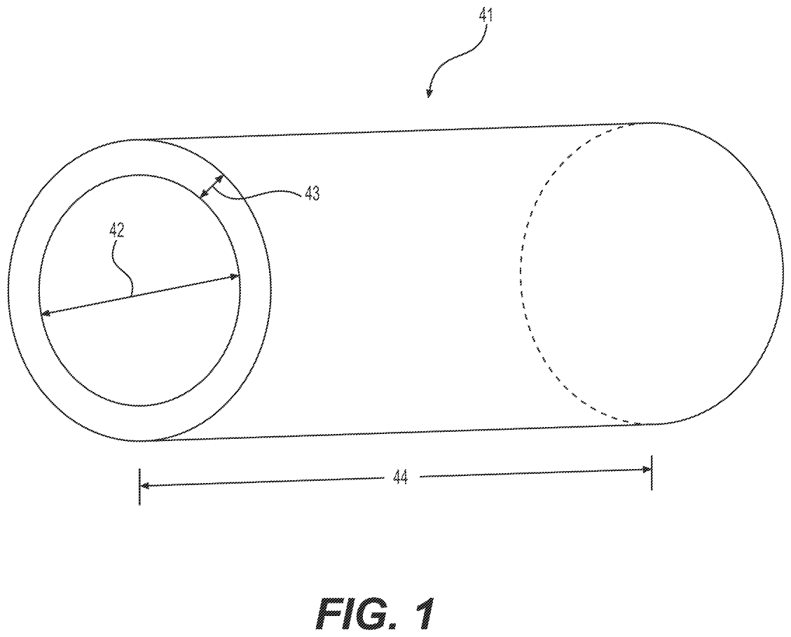

In an aspect of the present disclosure a sleeve comprises an outside diameter 41, an inside diameter 42, a thickness 43, and a length 44 and illustrated in FIG. 1. In an aspect, the inside diameter of a sleeve is equal to the size of the outside diameter of a tube 31 containing the kink. In another aspect, the inside diameter of the sleeve is smaller than the outside diameter of the tube containing the kink. In an aspect, the sleeve slides over the tube containing the kink. In another aspect the inside diameter 42 of the sleeve is about 4.32 mm. In another aspect, the inside diameter 42 is between 3 and 5 mm. In another aspect, the inside diameter 42 is between 3.5 and 5 mm. In another aspect, the inside diameter 42 is between 4 and 5 mm. In yet another aspect, the inside diameter 42 is between 4.2 and 5 mm.

In an aspect, the outside diameter 41 of the sleeve is about 6.35 mm. In another aspect, the outside diameter 41 is at least 5 mm. In another aspect, the outside diameter is between 5 and 12 mm. In another aspect, the outside diameter is between 10 and 12 mm. In another aspect, the outside diameter 41 is between 10 and 18 mm. In another aspect, the outside diameter 41 is between 5 and 18 mm. In another aspect, the outside diameter 41 is less than 18 mm. In another aspect, the outside diameter 41 is at least 0.5 mm larger than the inside diameter. In another aspect, the outside diameter 41 is at least 1.5 mm larger than the inside diameter. In another aspect, the outside diameter 41 is at least 50% larger than the inside diameter. In yet another aspect, the sleeve is at least 0.5 mm thick 43. In another aspect, the sleeve is between 0.5 and 1.5 mm thick 43.

In an aspect, the length 44 of the sleeve is about 12.7 mm. In another aspect, the length 44 is at least 6 mm. In another aspect, the length is between 4 and 500 mm. In another aspect, the length 44 is between 4 and 300 mm. In another aspect, the length is between 5 and 12 mm. In another aspect, the length 44 is between 5 and 20 mm. In another aspect, the length 44 is between 10 and 20 mm. In another aspect, the length is between 7.6 and 52 mm. In another aspect, the length 44 is less than 20 mm. In another aspect, the length 44 is at least 12 mm. In another aspect, the length of the sleeve is between 1.5 and 12-fold larger than the outside diameter of the tubing.

In an aspect of the present disclosure, the sleeve is used to remove kinks from medical tubing. In another aspect, the sleeve provided for is used to remove kinks from standard tubes in blood collection and transfusion kits. In another aspect, the sleeve is used to remove kinks from medical tubing selected from the group consisting of intravenous (IV) administration tubing, blood collection tubing, blood administration tubing, and hemodialysis and peritoneal dialysis. In an aspect, the sleeve is used to prevent kinks in a critical region of tubing (e.g., near equipment, bed frame, under a patient, etc.). In an aspect of the present disclosure, the tubing comprises a tubing clamp.

Sizing requirements of medical tubing is provided by International Standard ISO 3826. The ISO 3826 requires that transfer tubing have an internal diameter equal to or greater than 2.7 mm and a wall thickness 33 equal to or greater than 0.5 mm. In an aspect, the outside diameter of the tubing is about 3.7 mm. In another aspect, the outside diameter of the tubing is about 4.1 mm.

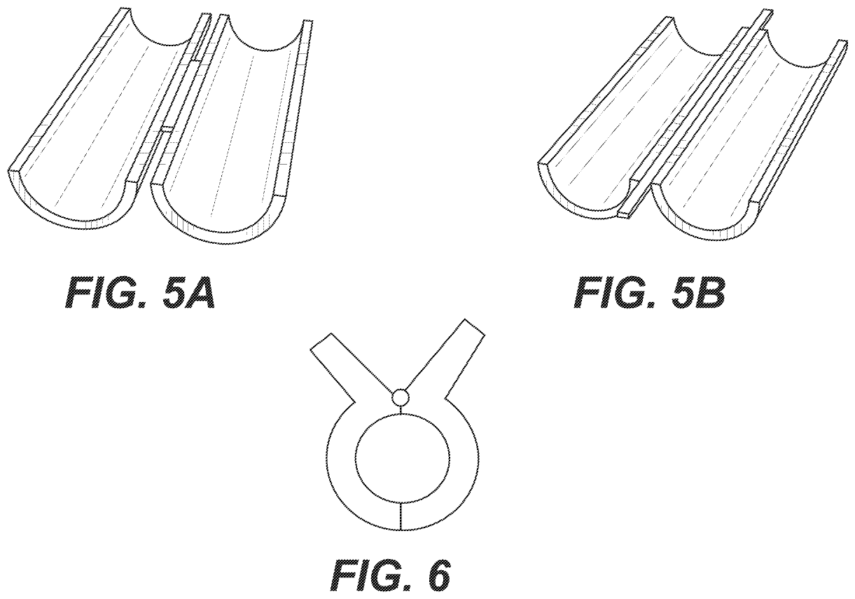

In an aspect, the inside diameter 42 of a sleeve is between 0.25 and 1 mm larger than the outside diameter 41 of the tubing. In another aspect, the inside diameter of a sleeve is at least 0.1 mm larger than the outside diameter of the tubing. In another aspect, the inside diameter of a sleeve is at most 1 mm larger than the outside diameter of the tubing. In yet another aspect, the inside diameter of the sleeve is between 0.5 and 8% larger than the outside diameter of the tubing. In yet another aspect, the inside diameter of the sleeve is less than 10% larger than the outside diameter of the tubing. The relative diameter of the inside diameter 42 of the sleeve and the outside diameter 31 of the tubing depends on the rigidity of the sleeve. A flexible sleeve can be stretched slightly allowing for a smaller ratio. Further, since the tubing can also be stretched, reducing the outside diameter 31, the inside diameter 42 of the sleeve can be the same size as the (un-stretched) outside diameter 31 of the tubing or slightly smaller. In yet another aspect, the outside diameter of the tubing is 4.1 mm and the inside diameter of a sleeve is 4.2 mm. In another aspect, the outside diameter of the tubing is 4.1 mm and the inside diameter of a sleeve is 4.5 mm. In an aspect of the present disclosure, the sleeve comprises two sections connected by a living hinge. A living hinge is a type of hinge made from an extension of the parent material to connect two larger sections. See generally, Chanbonpin, J., “Everything you need to know about living hinges (design, prototypes, and manufacturing)” (2017) (available on the internet at www.creativemechanisms.com/blog/everything-you-need-to-know-about-living-hinges), incorporated herein by reference in its entirety. In an aspect, the living hinge is along the longitudinal axis. In another aspect, the living hinge comprises material selected from the group consisting of polyethylene (PE), polypropylene (PP), acrylonitrile-butadiene-styrene (ABS), or polycarbonate. In another aspect, the living hinge comprises the same material as the sleeve. In another aspect, the living hinge is a continuous piece of material. In another aspect, the living hinge is segmented along the hinge line. In yet another aspect, the length of the living hinge is equal to the longitudinal length of the sleeve. In another aspect, the length of the living hinge is less than the longitudinal length of the sleeve (FIG. 5A). In a further aspect, the length of the living hinge is greater than the longitudinal length of the sleeve (FIG. 5B).

In an aspect of the present disclosure, the sleeve comprising a living hinge is capable of rotating 180 degrees or more. In another aspect the living hinge comprises two stops that serve to arrest rotation at 180 degrees. In this case, the living hinge comprises three unique pieces (triple hinge). In another aspect, the living hinge is capable of rotating between 0 and 180 degrees. In another aspect, the living hinge is capable of rotating greater than 90 degrees. In another aspect, the living hinge is a butterfly living hinge. A butterfly hinge, once opened past a certain angle, will remain in that position until moved. In another aspect, the living hinge is a double hinge. Double hinges comprise two strait hinges separated by a narrow landing section. These hinges allow for a 360 degree rotation.

In another aspect, the sleeve comprises two sections connected by a traditional hinge. In another aspect, the traditional hinge comprising multiple parts.

In an aspect of the present disclosure, a sleeve comprises a clip comprising a first edge portion and a second edge portion, lugs aligned with a spring intermediate the lugs, and a pin placed through the holes in the lugs. The first edge portion is connected to the second edge portion when the spring is not activated and separated from the second edge portion when the spring is activated. The first and second edge bend in a direction away from the gripping arm when the spring is activated, as shown in FIG. 6.

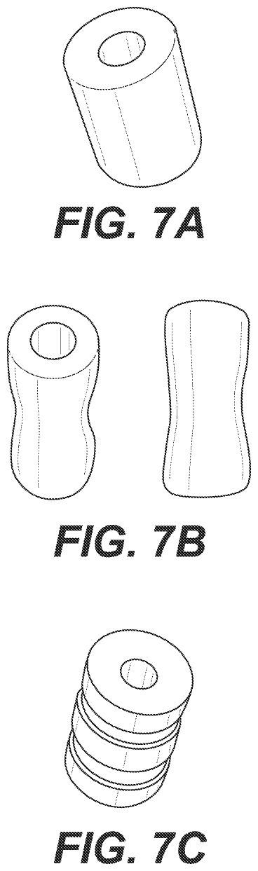

In an aspect of the present disclosure, the shape of the sleeve allows for secure handling. In another aspect, the sleeve has an hourglass shape to allow two fingers to be positioned at the smaller diameter (FIG. 7B). In another aspect, the sleeve has raised features as shown in FIG. 7C.

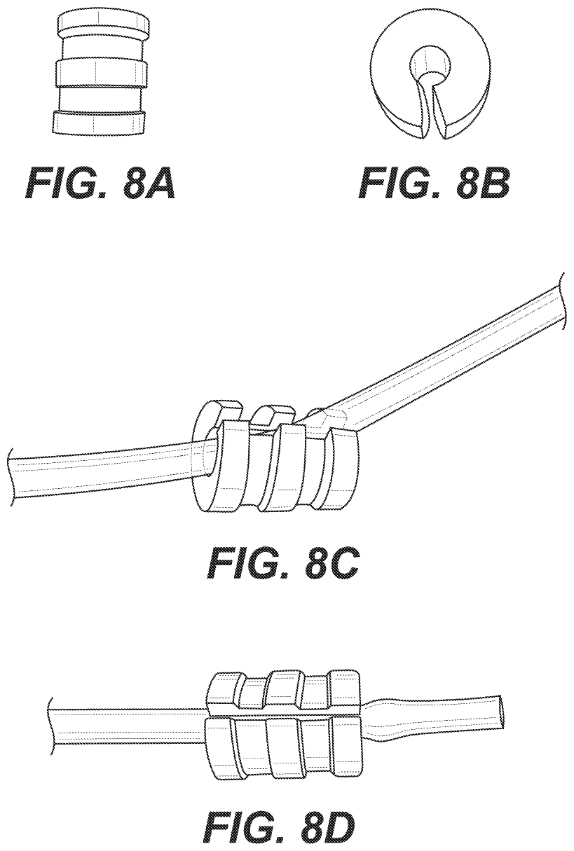

In an aspect of the present disclosure, the sleeve comprises an opening along its length 44, to allow the sleeve to slide over the kinked tubing (FIG. 8B to 8D). In an aspect, the opening is at least as wide as the kink. In another aspect, the opening is at least twice as wide as tube thickness 33. In another aspect, the opening is between 0.9 and 2 mm. In another aspect, the opening is between 0.9 and 1.1 mm. In another aspect, the opening is between 1.1 and 1.5 mm. In yet another aspect, the opening is between 1.5 and 2 mm. In another aspect, the opening is at least 0.9, 1.0, 1.2, 1.4, 1.5, 1.6, 1.8, or 2.0 mm. In yet another aspect, the opening is about 0.9, 1.0, 1.2, 1.4, 1.5, 1.6, 1.8, or 2.0 mm.

Methods of the present disclosure provide for, and include, removing a kink in a tube comprising: sliding a sleeve comprising a cylindrical inner diameter along the tube. In another aspect, the method comprises heating the sleeve while on the tubing. In another aspect, the heating time is between 1 and 60 mins. In another aspect, the heating time is at least 10 mins. In another aspect, the method comprises sliding the sleeve over the kink and allowing the sleeve to remain in place for a period of time. In an aspect, the sleeve is placed over the kink for seconds (secs), minutes (mins), or hours (hrs). In another aspect, the sleeve is placed over the kink for between 1 and 90 mins. In another aspect, the sleeve is placed over the kink indefinitely. In another aspect, the method further comprises placing the sleeve over the kink by sliding and rotating the sleeve. In another aspect, the sleeve is heated prior to placing it on the tubing. In another aspect, the tubing is heated. In yet another aspect, the sleeve is clamped on and rotated around the kink.

The present disclosure provides for restoring the flow of fluid in a tube comprising removing a kink in a tube comprising: sliding a sleeve comprising a cylindrical inner diameter along the tube. In an aspect, the flow is restored by 90% compared to the flow prior to kink removal. In an aspect, the flow is restored by 95% compared to the flow prior to kink removal. In an aspect, the flow is restored by 97% compared to the flow prior to kink removal. In an aspect, the flow is restored by 99% compared to the flow prior to kink removal. In another aspect, the flow is restored by at least 50% compared to the flow prior to kink removal. In another aspect, the flow is restored by at least 60% compared to the flow prior to kink removal. In another aspect, the flow is restored by at least 70% compared to the flow prior to kink removal. In another aspect, the flow is restored by at least 80% compared to the flow prior to kink removal. In another aspect, the flow is restored by 100% compared to the flow prior to kink removal. In yet another aspect, the flow is restored by between 20 and 100% compared to the flow prior to kink removal. In an aspect, the restoration of flow is at ambient pressure.

In another aspect of the present disclosure, the method comprises removing a kink in a tube by placing a sleeve comprising a living hinge over the tube comprising the kink.

Methods of the present disclosure provide for, and include, preventing a kink in a tube comprising: sliding a sleeve comprising a cylindrical inner diameter along the tube. In an aspect, the sleeve protects the tubing from bending.

The present disclosure provides for a kit comprising a blood bag connected to tubing and a sleeve threaded on the tubing.

As used herein the term “about” refers to ±10%.

As used herein the term “less than” refers to a smaller amount and an amount greater than zero.

The terms “comprises,” “comprising,” “includes,” “including,” “having,” and their conjugates mean “including but not limited to.”

The term “consisting of” means “including and limited to.”

The term “consisting essentially of” means that the composition, method or structure can include additional ingredients, steps and/or parts, but only if the additional ingredients, steps and/or parts do not materially alter the basic and novel characteristics of the claimed composition, method or structure.

As used herein, the singular forms “a,” “an,” and “the” include plural references unless the context clearly dictates otherwise. For example, the term “a compound” or “at least one compound” can include a plurality of compounds, including mixtures thereof.

As used herein, the term “blood” refers to whole blood, leukoreduced RBCs, platelet reduced RBCs, and leukocyte and platelet reduced RBCs. The term blood further includes packed red blood cells, platelet reduced packed red blood cells, leukocyte reduced packed red blood cells, and leukocyte and platelet reduced packed red blood cells.

As used herein, the term “rigid” means sleeve is not bendable. A rigid sleeve may have a flexural modulus value of greater than 2 GPa. In another aspect, a rigid sleeve has a flexural modulus value of greater than 0.5 GPa.

As used herein, the term “semi-rigid” means the sleeve is stiff but bendable when force is applied. A semi-rigid sleeve can have a flexural modulus value of between 0.1 and 0.5 GPa.

As used herein, the term “flexible” means the sleeve is capable of bending easily without breaking. A flexible sleeve can have a flexural modulus value of between 0.0001 and 0.1 GPa. In another aspect, a flexible sleeve has a flexural modulus value of less than 0.1 GPa. In yet another aspect, a flexible sleeve has a flexural modulus value of about 0.01 GPa.

The present disclosure provides for the following embodiments:

Embodiment 1. A device for restoring flow to a kinked tubing comprising a sleeve having cylindrical inner surface that is larger than the outside diameter of the tubing and said sleeve is slide-able along said tubing to compress said kinked tubing perpendicular to the kink.

Embodiment 2. The device of embodiment 1, wherein said sleeve comprises a material selected from the group consisting of polyethylene (PE), polypropylene (PP), metal, polyethylene terephthalate (PETG), polyvinyl chloride (PVC), PC, acrylic, nylon, acrylonitrile-butadiene-styrene (ABS), polytetrafluoroethylene (PTFE or Teflon), polyvinylidene fluoride (PVDF), polyether ether ketone (PEEK), chlorinated polyvinyl chloride (CPVC), fluorinated ethylene propylene (FEP), perfluoroalkoxy alkanes (PFA), Polyimide, Acetal, polyetherimide, and Ultem. In another aspect, the sleeve comprises two or more materials selected from the group consisting of polyethylene (PE), polypropylene (PP), metal, polyethylene terephthalate (PETG), polyvinyl chloride (PVC), PC, acrylic, nylon, acrylonitrile-butadiene-styrene (ABS), polytetrafluoroethylene (PTFE or Teflon), polyvinylidene fluoride (PVDF), polyether ether ketone (PEEK), chlorinated polyvinyl chloride (CPVC), fluorinated ethylene propylene (FEP), perfluoroalkoxy alkanes (PFA), Polyimide, Acetal, polyetherimide, and Ultem.

Embodiment 3. The device of embodiment 1 or 2, wherein said tubing is medical tubing selected from the group consisting of intravenous (IV) administration tubing, blood collection tubing, blood administration tubing, and hemodialysis and peritoneal dialysis.

Embodiment 4. The device of any one of embodiments 1 to 3, wherein said kinked tubing further comprising a tubing clamp.

Embodiment 5. The device of any one of embodiments 1 to 4, wherein said sleeve is semi-rigid.

Embodiment 6. The device of any one of embodiments 1 to 4, wherein said sleeve comprises a flexural modulus value at least twice the flexural modulus value of the kinked tubing.

Embodiment 7. The device of any one of embodiments 1 to 4, wherein said sleeve comprises a flexural modulus of less than 0.1 GPa.

Embodiment 8. The device any one of embodiments 1 to 7, wherein said inside diameter 42 of said sleeve is less than 10% larger than said outside diameter of said tubing.

Embodiment 9. The device of any one of embodiments 1 to 8, wherein said inside diameter 42 of said sleeve is between 0.5 and 8% mm larger than said outside diameter 31 of said tubing.

Embodiment 10. The device of any one of embodiments 1 to 8, wherein said inside diameter 42 of said sleeve is between 0.25 and 1.0 mm larger than said outside diameter 31 of said tubing.

Embodiment 11. The device of any one of embodiments 1 to 7, wherein said sleeve comprises an outside diameter 41 of between 6 and 18 mm.

Embodiment 12. The device of any one of embodiments 1 to 7, wherein said sleeve comprises an outside diameter 41 of about 6.35 mm.

Embodiment 13. The device of any one of embodiments 1 to 8, wherein said sleeve comprises an inside diameter 42 of between 3 and 5 mm.

Embodiment 14. The device of embodiment 13, wherein said sleeve comprises an inside diameter 42 of about 4.23 mm.

Embodiment 15. The device of any one of embodiments 1 to 14, wherein said sleeve comprises an thickness 43 of at least 0.5 mm.

Embodiment 16. The device of any one of embodiments 1 to 15, wherein said sleeve comprises an outside diameter 41 at least 50% larger than an inside diameter.

Embodiment 17. The device of any one of embodiments 1 to 16, wherein said sleeve comprises a length of between 4 and 52 mm.

Embodiment 18. The device of any one of embodiments 1 to 16, wherein said sleeve comprises a length of between 1.5 and 12-fold larger than the outside diameter of said tube.

Embodiment 19. The device of any one of embodiments 1 to 17, wherein said length is about 12.7 mm.

Embodiment 20. The device of any one of embodiments 1 to 19, wherein said sleeve comprises an outer surface comprising a feature selected from the group consisting of stippling, ribbing and knurling.

Embodiment 21. The device of embodiment 20, wherein said knurling is diamond knurling or straight diagonal knurling.

Embodiment 22. The device of any one of embodiments 1 to 19, wherein said sleeve comprises an outer surface comprising raised features.

Embodiment 23. The device of any one of embodiments 1 to 22, wherein said sleeve comprises an outer shape selected from the group consisting of a square, a triangle, a rectangle, a cylinder, a hexagon, an equilateral, a quadrilateral, a pentagon, and a trapezoid.

Embodiment 24. The device of any one of embodiments 1 to 23, wherein said sleeve comprises two sections connected by a living hinge along the longitudinal axis.

Embodiment 25. The device of embodiment 24, wherein said at least one section is capable of rotating at angles of 180 degrees or greater.

Embodiment 26. A method of removing a kink in a tube comprising: sliding a sleeve comprising a cylindrical inner diameter along said tube.

Embodiment 27. The method of embodiment 26, further comprising heating said sleeve on said tube.

Embodiment 28. The method of embodiment 26, wherein said sliding comprises covering said kink with said sleeve for a period of time between 1 second and 1 hour.

Embodiment 29. The method of any one of embodiments 26 to 28, wherein said sliding comprises covering said kink for at least 1 hr.

Embodiment 30. The method of any one of embodiments 26 to 29, further comprising rotating said sleeve.

Embodiment 31. The method of any one of embodiments 26 to 30, wherein said sleeve comprises a material selected from the group consisting of polyethylene (PE), polypropylene (PP), metal, polyethylene terephthalate (PETG), polyvinyl chloride (PVC), PC, acrylic, nylon, acrylonitrile-butadiene-styrene (ABS), polytetrafluoroethylene (PTFE or Teflon), polyvinylidene fluoride (PVDF), polyether ether ketone (PEEK), chlorinated polyvinyl chloride (CPVC), fluorinated ethylene propylene (FEP), perfluoroalkoxy alkanes (PFA), Polyimide, Acetal, polyetherimide, and Ultem. In another aspect, the sleeve comprises two or more materials selected from the group consisting of polyethylene (PE), polypropylene (PP), metal, polyethylene terephthalate (PETG), polyvinyl chloride (PVC), PC, acrylic, nylon, acrylonitrile-butadiene-styrene (ABS), polytetrafluoroethylene (PTFE or Teflon), polyvinylidene fluoride (PVDF), polyether ether ketone (PEEK), chlorinated polyvinyl chloride (CPVC), fluorinated ethylene propylene (FEP), perfluoroalkoxy alkanes (PFA), Polyimide, Acetal, polyetherimide, and Ultem.

Embodiment 32. The method of any one of embodiments 26 to 31, wherein said tubing is medical tubing selected from the group consisting of intravenous (IV) administration tubing, blood collection tubing, blood administration tubing, and hemodialysis and peritoneal dialysis.

Embodiment 33. The method of any one of embodiments 26 to 32, wherein said sleeve is semi-rigid.

Embodiment 34. The method of any one of embodiments 26 to 32, wherein said sleeve is rigid.

Embodiment 35. The method of any one of embodiments 26 to 32, wherein said sleeve comprises a flexural modulus of less than 0.1 GPa.

Embodiment 36. The method of any one of embodiments 26 to 35, wherein said inside diameter 42 is larger than the outside diameter 31 of said tubing.

Embodiment 37. The method of embodiment 36, wherein said inside diameter 42 of said sleeve is less than 10% mm larger than said outside diameter of said tubing.

Embodiment 38. The method of embodiment 36, wherein said inside diameter 42 of said sleeve is between 0.5 and 8% mm larger than said outside diameter of said tube.

Embodiment 39. The method of embodiment 36, wherein said inside diameter 42 of said sleeve is equal to said outside diameter of said tubing.

Embodiment 40. The method of any one of embodiments 26 to 36, wherein said sleeve comprises an outside diameter 41 of 6.35 mm.

Embodiment 41. The method of any one of embodiments 26 to 38, wherein said inside diameter 42 is 4.23 mm.

Embodiment 42. The method of embodiment 26, wherein said sleeve comprises an outside diameter 41 at least 1.5 mm larger than said inside diameter.

Embodiment 43. The method of any one of embodiments 26 to 41, wherein said sleeve comprises an outside diameter 41 at least 50% larger than said inside diameter.

Embodiment 44. The method of any one of embodiments 26 to 43, wherein said sleeve comprises a length of between 4 and 52 mm.

Embodiment 45. The method of any one of embodiments 26 to 43, wherein said sleeve comprises a length of between 1.5 and 12 fold larger than the outside diameter of said tube.

Embodiment 46. The method of any one of embodiments 26 to 43, wherein said sleeve comprises a length of between 1.5 and 12 fold larger than the outside diameter of said tube.

Embodiment 47. The method of embodiment 44, wherein said sleeve comprises a length of about 12.7 mm.

Embodiment 48. The method of any one of embodiments 26 to 47, wherein said sleeve comprises an outer surface comprising a feature selected from the group consisting of stippling, ribbing and knurling.

Embodiment 49. The method of any one of embodiments 26 to 48, wherein said knurling is diamond knurling or straight diagonal knurling.

Embodiment 50. The method of any one of embodiments 26 to 49, wherein said sleeve comprises an outer surface comprising raised features.

Embodiment 51. The method of any one of embodiments 26 to 50, wherein said sleeve comprises an outer shape selected from the group consisting of a square, a triangle, a rectangle, a cylinder, a hexagon, an equilateral, a quadrilateral, a pentagon, and a trapezoid.

Embodiment 52. The method of any one of embodiments 26 to 51, wherein said sleeve comprises an inner diameter comprising a chamfer.

Embodiment 53. A device for restoring flow to a kinked tubing comprising a sleeve having a cylindrical inner surface equal to or smaller than the outside diameter of the tubing and said sleeve is capable of compressing said kinked tubing perpendicular to the kink.

Embodiment 54. The device of embodiment 53, wherein said sleeve comprises polyethylene (PE), polypropylene (PP), metal, polyethylene terephthalate (PETG), polyvinyl chloride (PVC), PC, acrylic, nylon, acrylonitrile-butadiene-styrene (ABS). a material selected from the group consisting of polyethylene (PE), polypropylene (PP), metal, polyethylene terephthalate (PETG), polyvinyl chloride (PVC), PC, acrylic, nylon, acrylonitrile-butadiene-styrene (ABS), polytetrafluoroethylene (PTFE or Teflon), polyvinylidene fluoride (PVDF), polyether ether ketone (PEEK), chlorinated polyvinyl chloride (CPVC), fluorinated ethylene propylene (FEP), perfluoroalkoxy alkanes (PFA), Polyimide, Acetal, polyetherimide, and Ultem. In another aspect, the sleeve comprises two or more materials selected from the group consisting of polyethylene (PE), polypropylene (PP), metal, polyethylene terephthalate (PETG), polyvinyl chloride (PVC), PC, acrylic, nylon, acrylonitrile-butadiene-styrene (ABS), polytetrafluoroethylene (PTFE or Teflon), polyvinylidene fluoride (PVDF), polyether ether ketone (PEEK), chlorinated polyvinyl chloride (CPVC), fluorinated ethylene propylene (FEP), perfluoroalkoxy alkanes (PFA), Polyimide, Acetal, polyetherimide, and Ultem.

Embodiment 55. The device of embodiment 53, wherein said tubing is medical tubing selected from the group consisting of intravenous (IV) administration tubing, blood collection tubing, blood administration tubing, and hemodialysis and peritoneal dialysis.

Embodiment 56. The device of embodiment 53, wherein said kinked tubing further comprises a clamp.

While the present disclosure has been described with reference to particular embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the scope of the present disclosure.

Therefore, it is intended that the present disclosure not be limited to the particular embodiments disclosed as the best mode contemplated for carrying out the present disclosure, but that the present disclosure will include all embodiments falling within the scope and spirit of the appended claims.

### EXAMPLES

### Example 1: Manufacture and Removal of a Tubing Kink

To manufacture a kink, a piece of PVC tubing meeting the sizing requirements of ISO 3826 is threaded through a plastic clamp (Halkey-Roberts #C340TCSPX). The clamp is closed and heat is applied to the tubing with a heat-shrink gun to exacerbate the kink in the PVC tubing caused by the clamp.

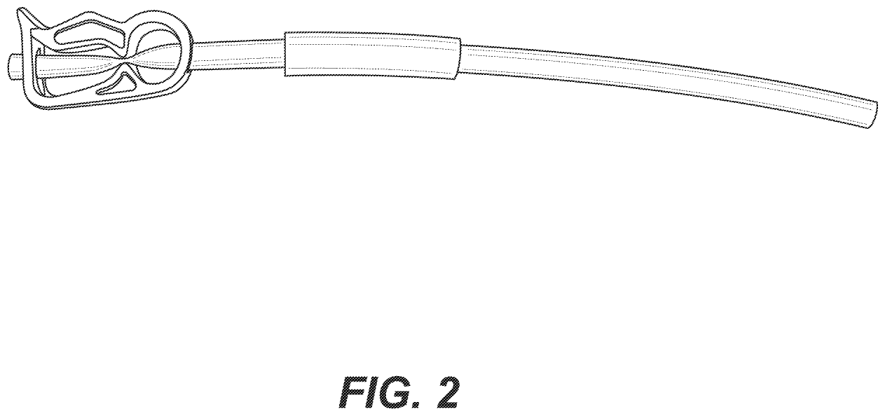

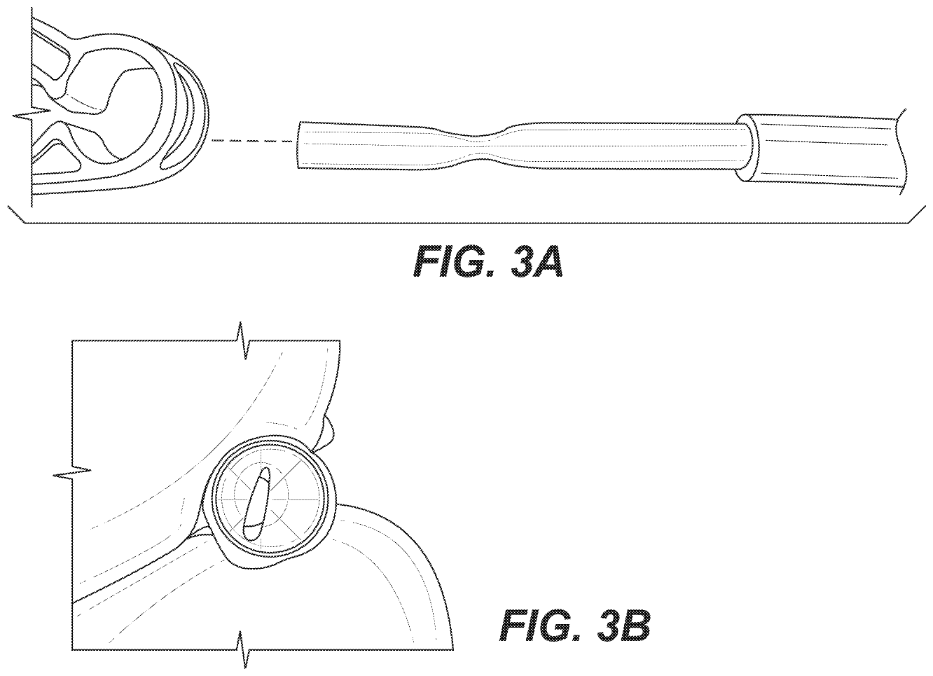

A piece of Poly-Flo® semi-rigid polyethylene tubing having an outside diameter of about 6.35 mm, an inside diameter of about 4.32 mm, and a length of 12.7 mm, is placed over the PVC tubing near the clamp (FIG. 2). The clamp is removed, leaving a kink in the tube. The tube is unusable due to the reduction of the inner diameter and restriction of fluid flow therein (FIGS. 3A and 3B).

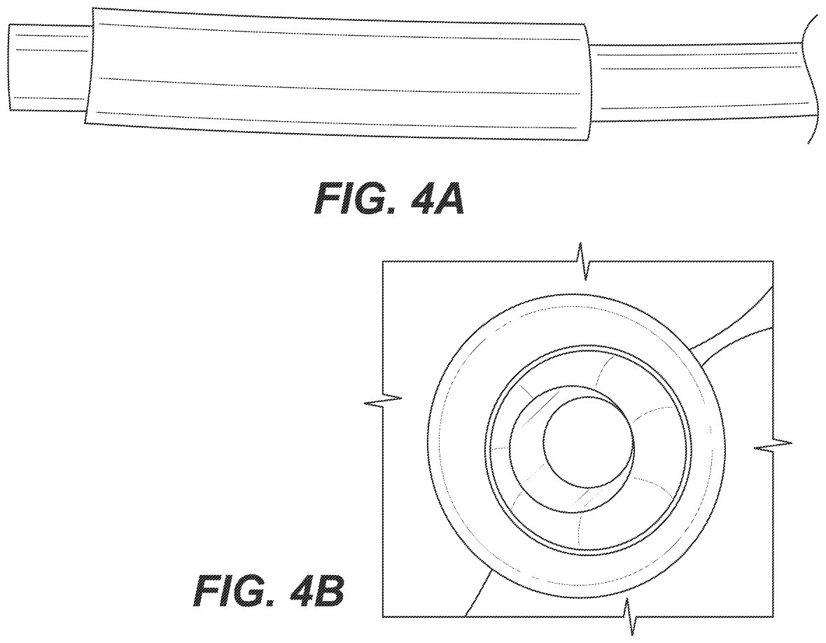

The kink is removed by sliding the sleeve over the kink (FIGS. 4A and 4B) compressing the tubing perpendicular to the kink and restoring flow.

We claim:

1. A device for restoring flow to a kinked tubing comprising a sleeve having cylindrical inner surface that is larger than the outside diameter of the tubing and said sleeve is slide-able along said tubing to compress said kinked tubing perpendicular to the kink.

2. The device of claim 1, wherein said sleeve comprises a material selected from the group consisting of polyethylene (PE), polypropylene (PP), metal, polyethylene terephthalate (PETG), polyvinyl chloride (PVC), PC, acrylic, nylon, acrylonitrile-butadiene-styrene (ABS), polytetrafluoroethylene (PTFE or Teflon), polyvinylidene fluoride (PVDF), polyether ether ketone (PEEK), chlorinated polyvinyl chloride (CPVC), fluorinated ethylene propylene (FEP), perfluoroalkoxy alkanes (PFA), Polyimide, Acetal, polyetherimide, and Ultem. In another aspect, the sleeve comprises two or more materials selected from the group consisting of polyethylene (PE), polypropylene (PP), metal, polyethylene terephthalate (PETG), polyvinyl chloride (PVC), PC, acrylic, nylon, acrylonitrile-butadiene-styrene (ABS), polytetrafluoroethylene (PTFE or Teflon), polyvinylidene fluoride (PVDF), polyether ether ketone (PEEK), chlorinated polyvinyl chloride (CPVC), fluorinated ethylene propylene (FEP), perfluoroalkoxy alkanes (PFA), Polyimide, Acetal, polyetherimide, and Ultem.

3. The device of claim 1, wherein said tubing is medical tubing selected from the group consisting of intravenous (IV) administration tubing, blood collection tubing, blood administration tubing, and hemodialysis and peritoneal dialysis.

4. The device of claim 1, wherein said kinked tubing further comprising a tubing clamp.

5. The device of claim 1, wherein said sleeve is flexible.

6. The device claim 1, wherein said inside diameter 42 of said sleeve is less than 10% larger than said outside diameter of said tubing.

7. The device of claim 1, wherein said sleeve comprises an outside diameter 41 of between 6 and 18 mm.

8. The device of claim 1, wherein said sleeve comprises an inside diameter 42 of between 3 and 5 mm.

9. The device of claim 1, wherein said sleeve comprises an outer surface comprising a feature selected from the group consisting of stippling, ribbing and knurling.

10. The device of claim 9, wherein said knurling is diamond knurling or straight diagonal knurling.

11. The device of claim 1, wherein said sleeve comprises an outer surface comprising raised features.

12. A method of removing a kink in a tube comprising: sliding a sleeve comprising a cylindrical inner diameter along said tube.

13. The method of claim 12, further comprising heating said sleeve on said tube.

14. The method of claim 12, wherein said sleeve comprises a material selected from the group consisting of polyethylene (PE), polypropylene (PP), metal, polyethylene terephthalate (PETG), polyvinyl chloride (PVC), PC, acrylic, nylon, acrylonitrile-butadiene-styrene (ABS), polytetrafluoroethylene (PTFE or Teflon), polyvinylidene fluoride (PVDF), polyether ether ketone (PEEK), chlorinated polyvinyl chloride (CPVC), fluorinated ethylene propylene (FEP), perfluoroalkoxy alkanes (PFA), Polyimide, Acetal, polyetherimide, and Ultem. In another aspect, the sleeve comprises two or more materials selected from the group consisting of polyethylene (PE), polypropylene (PP), metal, polyethylene terephthalate (PETG), polyvinyl chloride (PVC), PC, acrylic, nylon, acrylonitrile-butadiene-styrene (ABS), polytetrafluoroethylene (PTFE or Teflon), polyvinylidene fluoride (PVDF), polyether ether ketone (PEEK), chlorinated polyvinyl chloride (CPVC), fluorinated ethylene propylene (FEP), perfluoroalkoxy alkanes (PFA), Polyimide, Acetal, polyetherimide, and Ultem.

15. The method of claim 12, wherein said inside diameter 42 of said sleeve is less than 10% mm larger than said outside diameter of said tubing.

16. The method of claim 15, wherein said inside diameter 42 of said sleeve is equal to said outside diameter of said tubing.

17. The method of claim 12, wherein said sleeve comprises a length of between 4 and 52 mm.

18. The method of claim 12, wherein said sleeve comprises an outer surface comprising raised features.

19. A device for restoring flow to a kinked tubing comprising a sleeve having a cylindrical inner surface equal to or smaller than the outside diameter of the tubing and said sleeve is capable of compressing said kinked tubing perpendicular to the kink.

20. The device of claim 19, wherein said sleeve comprises polyethylene (PE), polypropylene (PP), metal, polyethylene terephthalate (PETG), polyvinyl chloride (PVC), PC, acrylic, nylon, acrylonitrile-butadiene-styrene (ABS). a material selected from the group consisting of polyethylene (PE), polypropylene (PP), metal, polyethylene terephthalate (PETG), polyvinyl chloride (PVC), PC, acrylic, nylon, acrylonitrile-butadiene-styrene (ABS), polytetrafluoroethylene (PTFE or Teflon), polyvinylidene fluoride (PVDF), polyether ether ketone (PEEK), chlorinated polyvinyl chloride (CPVC), fluorinated ethylene propylene (FEP), perfluoroalkoxy alkanes (PFA), Polyimide, Acetal, polyetherimide, and Ultem. In another aspect, the sleeve comprises two or more materials selected from the group consisting of polyethylene (PE), polypropylene (PP), metal, polyethylene terephthalate (PETG), polyvinyl chloride (PVC), PC, acrylic, nylon, acrylonitrile-butadiene-styrene (ABS), polytetrafluoroethylene (PTFE or Teflon), polyvinylidene fluoride (PVDF), polyether ether ketone (PEEK), chlorinated polyvinyl chloride (CPVC), fluorinated ethylene propylene (FEP), perfluoroalkoxy alkanes (PFA), Polyimide, Acetal, polyetherimide, and Ultem.
